(19) United States
(12) Patent Application Publication
(10) Pub. No.: US 2025/0375157 A1
(43) Pub. Date: Dec. 11, 2025

(54) RESPIRATORY-CARDIAC ARREST DETECTION AND DRUG OVERDOSE TREATMENT SYSTEM

(71) Applicant: John Sassano, Daytona Beach, FL (US)

(72) Inventor: John Sassano, Daytona Beach, FL (US)

(21) Appl. No.: 19/261,283

(22) Filed: Jul. 7, 2025

Related U.S. Application Data
(62) Division of application No. 18/736,149, filed on Jun. 6, 2024, now abandoned.

(51) Int. Cl. A61B 5/00; A61B 5/0205; A61B 5/11; A61M 5/172

(52) U.S. Cl. CPC A61B 5/4839; A61B 5/0205; A61B 5/4845; A61B 5/7282; A61B 5/741; A61B 5/742; A61B 5/746; A61B 5/747; A61B 5/1112; A61M 5/1723

(57) ABSTRACT

The invention system is a wearable device that detects an impending or onset of cardio-respiratory arrest and remediates the condition by injecting an appropriate reversal drug depending upon the drug that triggered the arrest. It can detect and remediate arrests caused by drugs such as opioids, benzodiazepine or alcohol. Using self-test and other checks, it ensures that the system is operationally ready and that no near-expiration or expired drug is administered.

### CROSS-REFERENCE TO RELATED APPLICATIONS

The present application claims priority to U.S. Provisional Application No. 18/736,149 filed Jun. 6, 2024, titled “RESPIRATORY-CARDIAC ARREST DETECTION AND DRUG OVERDOSE TREATMENT SYSTEM,” which is hereby incorporated by reference in its entirety.

### TECHNICAL FIELD

The invention system is a self-contained, wearable device that monitors vital signs for an indication of cardio-respiratory arrest and applies remediation via the injection of arrest-reversing drugs.

BACKGROUND

Although a near-epidemic of deaths, worldwide and in the United States, has been due to opioid-related overdosing, other drugs and alcohol can cause similar collapse of cardio-respiratory function. Often, by the time it is clear that a person is suffering an overdose that has arrested cardiac and respiratory function, there is a chance of revival by injecting drugs that reverse the situation. However, time is of the essence in such cases.

Drugs such as Naloxone, Flumazenil and Metadoxine are known to reverse cardio-respiratory arrest caused by opioids, benzodiazepine and alcohol, respectively. Time is the key factor. Ideally, as soon as someone's vitals show cardio-respiratory arrest in progress, that's the best time to inject a reversal drug, such as those above.

By monitoring vitals such as pulse rate, respiration, blood pressure, CO2, oxygen saturation, ECG and muscle activity, one is able to see the onset of arrest often before it is likely to become irreversible. Then, if a reversal drug is immediately at hand, with a means to deliver it, successful remediation is highly likely.

People who are at risk of cardio-respiratory arrest due to opioid, benzodiazepine or alcohol consumption would be better protected from fatal consequences if they had a system that constantly monitored their vitals, was capable of detecting and identifying an impending arrest, and was capable of then administering a reversal drug well before irreversibility.

### SUMMARY OF THE INVENTION SYSTEM

The invention system herein disclosed is a wearable device that can be attached to an arm or other appendage such that one surface is in contact with the skin below. Using one or more sensors, operative to monitor and measure vital functions, a microcontroller contained within the device can compare readings in real-time or near real-time to predetermined safe ranges and determine if one or more vital functions is amiss. Once the readings are determined to fall below a safe level, and are indicative of an impending arrest, the device initiates a remediation response.

The response comprises an audible alarm, a displayed message, a voice message, initiation of the injection of a reversal drug, injection of the drug, continued vital-sign monitoring, and a call to a first-responder group that includes emergency description and GPS location. Using other wireless communications means, the device may also interface with a smartphone, laptop, desktop or other form of computer system. The system also stores the vital-sign readings that triggered the remediation response which may be easily downloaded by responding emergency or medical personnel.

### BRIEF DESCRIPTION OF THE DRAWINGS

FIG. 1 shows an embodiment of the wearable device on a person's arm near the wrist.

FIG. 2 shows the subsystems and interrelationships of the functional blocks comprising the invention system.

FIG. 3 shows a three-dimensional inner view of the invention system showing the positions of functional blocks and external features.

FIG. 4 is a flow diagram of a program portion that directs power-level monitoring and determination of system power voltage status.

FIG. 5 is a flow diagram of a program portion that checks expiration of reversal-drugs installed in the system and determines if drugs have expired prior to dispensing them.

FIG. 6 is a flow diagram that illustrates a program portion that provides a system-status alarm.

FIG. 7 is a flow diagram of a program portion that checks vital signs, determines if an arrest is impending, and, if so, triggers an arrest alarm and guides the remediation response and follow up.

FIG. 8 is a flow diagram of a program portion that governs the response to an arrest alarm invocation.

### DETAILED DESCRIPTION

A cardio-respiratory arrest can be caused by opioid, benzodiazepine, alcohol overconsumption, and other drugs. The resulting impact on bodily vital signs, such as pulse rate, blood pressure, CO2, oxygen saturation, and muscle activity is similar when arrest due to any of these substances is impending. However, different drugs are required to reverse the cardio-respiratory arrest condition depending on the drug that triggered it.

The invention system, exemplified in one of a variety of embodiments, is meant to continuously monitor a user's vitals, detect an impending cardio-respiratory arrest, and initiate a remediation response that reverses the impending or manifesting arrest.

It is a self-contained system, housed in a low-profile encasement, and mounted to an arm or other appendage using a strapping fixture. One surface of the encasement is in contact with the skin. Inside the encasement are one or more sensors operative to detect one or more vital signs and convey the results to a controller subsystem. The controller subsystem, comprising program and data storage, central processor, and input-output functional blocks, executes one or more programs operative to input sensor data and compare vital-sign readings to a predetermined range of normal-range values.

If the sensor inputs provide data that falls outside the safe limits of values, the invention system responds by asserting an arrest alarm condition, and initiating the deployment of an injector and the injection of a reversal drug into a user. While responding to the arrest condition, the invention system continues to monitor the vital signs to detect when vitals have returned to a safe value range. Concurrently, the invention system contacts first responders providing a description of the emergency and GPS coordinates of the person. The invention system also stores the vital-sign values that initially triggered the arrest alarm and remediation response in its data storage such that responding emergency and/or medical personnel can download that vital-sign data. Events may be time stamped such that first responders can view the various events and be informed of the time at which the events occurred. Events may be defined as any biological event often used as an indicator for cardiac arrest.

The same invention system can be used for people subject to cardio-respiratory arrest due to use of opioids, benzodiazepine or alcohol. The different reversal drugs are kept in bladders or cartridges that will dispense the fluid when compressed. The key difference between how the invention system is employed for, say, opioid-versus benzodiazepine-caused cardio-respiratory remediation are the reversal drugs contained in the bladder or cartridges, and any differences in vital-sign changes associated with arrest due to opioid-compared with benzodiazepine-induced arrest.

Since the system is self-contained, its power source is a battery which over time will drop in voltage level. At a predetermined level, S, which is chosen as slightly above the lowest level that still maintains correct system behavior, the system will initiate a status alarm, similar to that of a smoke detector whose batteries are nearing a too-low power level. That is, it will provide a periodic beep and display a battery-replacement-necessary message on its display subsystem. It will also place the system in standby until it detects that a battery has been replaced as indicated by a higher voltage reading on the invention system's power bus.

The drug-containing bladders or cartridges have a code on their side that indicates expiration date. When the bladder or cartridge is installed in the invention system, a code reading subsystem captures the expiration date and uses that to periodically check expiration status. At some predetermined period before expiration, the system will initiate a status alarm, like the low-battery alarm, but the display will have a replace expired-drug-cartridge message.

Ancillary to the vital-sign detection and remediation functions, the invention system also contains a GPS subsystem, a cellular communication subsystem and a wireless-communications subsystem. The GPS subsystem provides GPS coordinates at the time of arrest detection, and the wireless-communication subsystem enables the invention system to interface with another system, such as a smartphone or laptop computer. The cellular communications subsystem enables a call to a first-responder phone number.

With the foregoing as a high-level overview, the following is meant to provide a more in-depth description of the invention system and its subsystems. In addition, the following provides some exemplary flow diagrams of program portions that support vital-sign detection, system status condition detection, alarm responses and arrest responses.

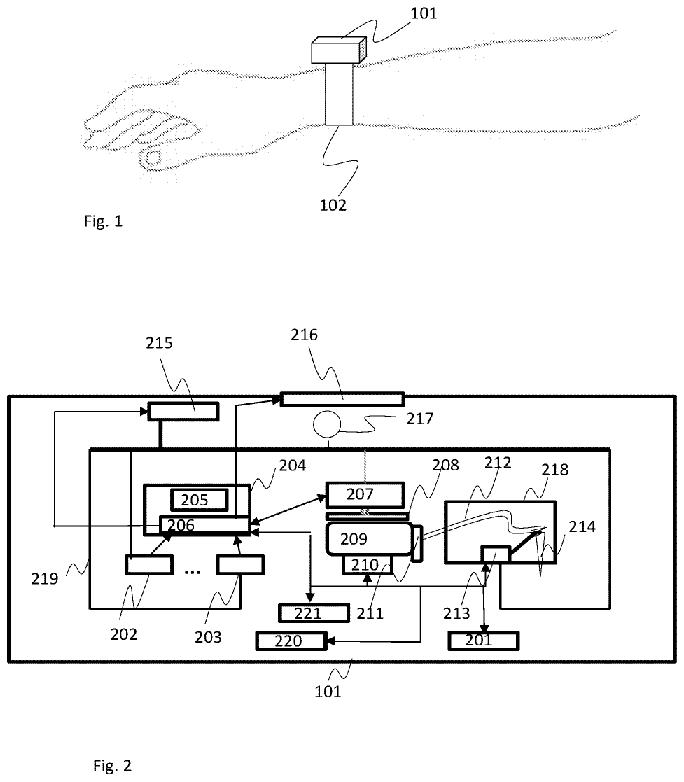

FIG. 1 shows an embodiment of the invention system that sits on an arm, near the wrist. The invention system and its subsystems are all contained in encasement 101. The invention system is held in place by a strapping fixture 102.

FIG. 2 shows the subsystems comprising the invention system all contained within the encasement 101. One or more sensors, 202 and 203, are operative to detect and measure specific vital signs, such as respiration, pulse rate, and the like. The sensor data is input over an I/0 bus into a controller subsystem 204 comprising a central processor with program and data memory 205 and input-output (I/0) 206. Executing one or more programs, the controller subsystem compares sensor input data to predetermined safe value ranges. The controller, via the power bus, monitors bus voltage and compares it to a predetermined value, S. The controller also receives input from a code reader 210 with the expiration date data of a reversal-drug bladder or cartridge 209.Portions of the controller's program or programs use the power-bus levels and expiration-date data to determine if low-battery or drug-expiration conditions are impending. If so, a system-status alarm is invoked wherein a periodic beeping and display message alert the user to either a low-battery or expired-drug status.

Where the controller, based on sensor data, concludes that a cardio-respiratory arrest is impending or manifesting, it asserts an arrest alarm. Rather than the beeping of the system-status alarm, the arrest alarm produces a piercing, continuous tone or warbling tone through a transducer system 215. It also displays emergency alarm messages on its display subsystem 216.

Concurrent with the arrest alarm invocation, the controller sends a deployment command to an injector subsystem's (218) injector deployment subsystem 213 which propels an injection need 214 down through an aperture and into the epidermis of the user. Only after the controller has received return data from the injector subsystem that the needle has been deployed will it then initiate injection by sending a signal to a depressor subsystem 207 (e.g., such as one including an Archimedes device) which compresses, using a compressing structure (208), the drug bladder or cartridge 209 pushing the drug fluid through leak-proof interface 211 to conveyance tube 212 to the needle 214. Only after the controller has received return data from the depressor subsystem that the bladder or cartridge has been compressed will it then initiate injection retraction essentially removing the needle from a user's skin.

In some embodiments, the Archimedes device allows for the reduction of length of the device by loading the Archimedes device with the reversal drug. The Archimedes device is turned to inject reversal drug. This removes the additional length required by a plunger mechanism, reducing the length of the device.

Vital-sign data that triggered the arrest alarm and remediation response will have been stored in data storage in the controller subsystem. A cellular radio subsystem 221 is prompted to call a first-responder emergency number to provide GPS coordinates as captured by GPS subsystem 201. A wireless-communications subsystem 220 may be used to interface the invention system to a smartphone or other computer device. This may also be used as a means for downloading the stored vital-signs data.

A power source 217 provides the power for all subsystems via a power bus 219. The controller subsystem sends, receives, or sends and receives data and commands via unidirectional and bidirectional data buses, as shown. The power source 217 may be any power source commonly used in the arts (e.g., a battery, rechargeable battery, etc.).

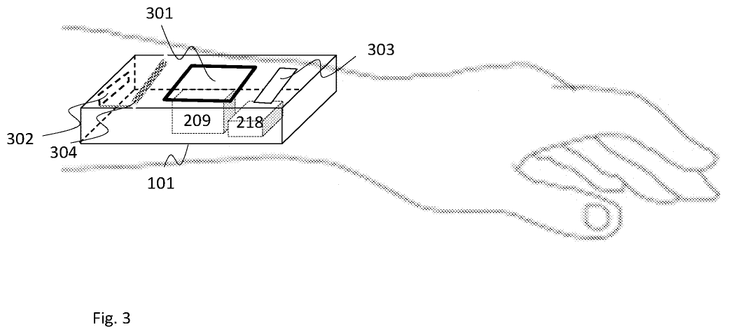

In FIG. 3, the encasement 101 is shown in place on a user's arm and the position of the injector subsystem 218 is shown as on the face of the encasement inside surface closest to the user's skin. This enables the injector to deploy the needle downward, through the encasement and into the user's skin. The bladder or cartridge 209 is shown near the injector subsystem 218 and an opening 301 on the upper face of the encasement facilitates installing new, full cartridges and removing spent or expired cartridges. A side opening 302 facilitates inserting or replacing the button-cell battery without having to remove the device from one's arm. A small area of apertures 304 lies adjacent to the transducer subsystem to allow sound to escape the encasement. And, a display subsystem window 303 is shown on the upper surface of the encasement allowing easily accessible message reading.

The transducer subsystem comprises a transducer, a digital-to-analog converter (DAC), and an audio amplifier, wherein a digital signal from the controller subsystem is conveyed to the transducer subsystem where the DAC converts the signal to an audio signal, it passes through the amplifier, and excites the transducer producing tones or voice messaging.

The injector subsystem comprises an electro-mechanical device operative to extend or retract its hollow injection needle, and provides status data back to the controller subsystem.

The depressor subsystem comprises an electro-mechanical device, such as a solenoid and plunger, which causes the plunger to push against the reversal-drum cartridge and compress it against a scaffold such that all reversal-drug fluid is conveyed, via a tube, to the injector subsystem's hollow injection needle.

The scaffold upon which the reversal-drug cartridge is seated provides a immovable base, a leak-proof interface between cartridge and fluid-conveyance tube, and has a code reader operative to read the code displayed on the reversal-drug cartridge which contains the expiration date.

FIGS. 1, 2 and 3 are meant to be exemplary and should not be read as limiting the scope of the invention system.

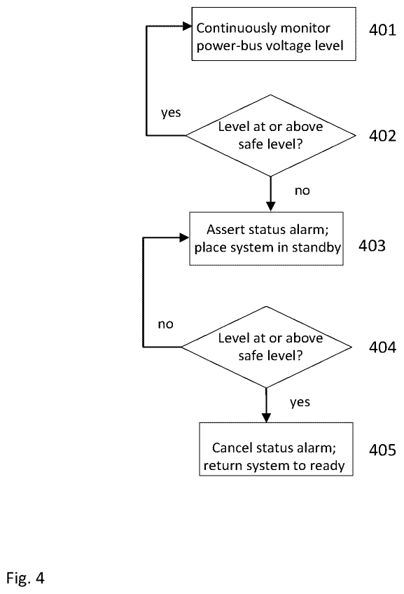

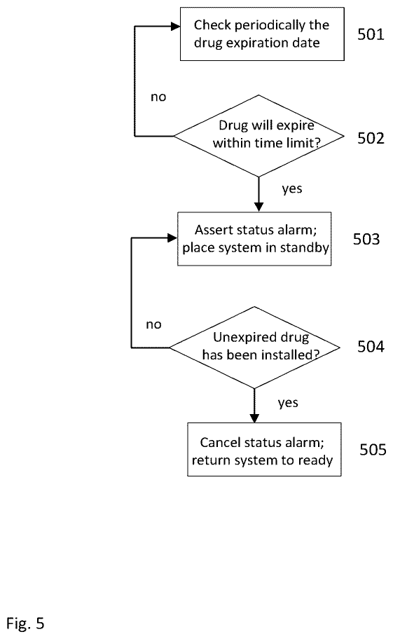

FIG. 4 is an exemplary flow diagram of a program portion for monitoring power-bus voltage level and ensuring that as the level nears a predetermined lower limit, S, that the system is placed in standby and the user is alerted to a changed system-status condition. As shown in 401 the power bus voltage level is continuously monitored. So long as it is at or above the limiting value, S, no action is taken by virtue of the conditional function 402. If the level has dropped below S, the controller asserts a status alarm 403 and preserves that alarm and standby condition 404 so long as the level is below S. Once it detects that the power-bus voltage level is above S, the controller cancels the status alarm and returns the system to ready state 405.

As used herein, the term “standby” refers to the continued monitoring and sensing of the users condition, without an intervention being performed by the system.

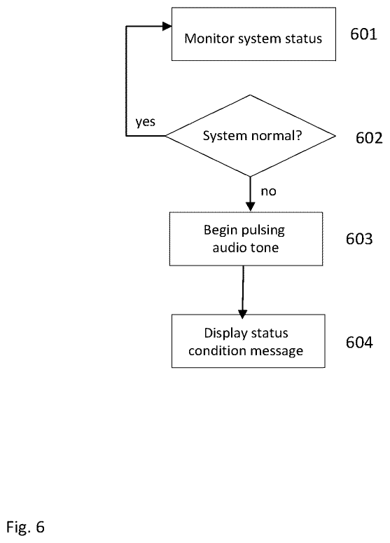

FIG. 5 is an exemplary flow diagram of a program portion for preventing use of an expired drug. Once, per each predetermined time period, the expiration date is checked 501 and if the drug will not expire within a predetermined time limit, no action is taken. If the drug will expire within the predetermined time lime 502 the controller asserts a status alarm 503 and places the system in standby. It then continues to check expiration date for the drug bladder or cartridge 504 and upon detecting a drug that will not expire within the predetermined time period, controller will cancel status alarm and return system to ready state 505.

FIG. 6 shows an exemplary flow diagram of a program portion that executes a status alarm after checking system status 601. If no alarm (e.g. system normal and no sensor failure is detected) no action is taken. If system-status alarm assertion is detected 602 or the sensors detect below-normal values, the controller commands the transducer subsystem to produce the audible beeping tone 603 and commands the display subsystem to provide the appropriate message 604 (e.g., battery needs replacement or expired drug).

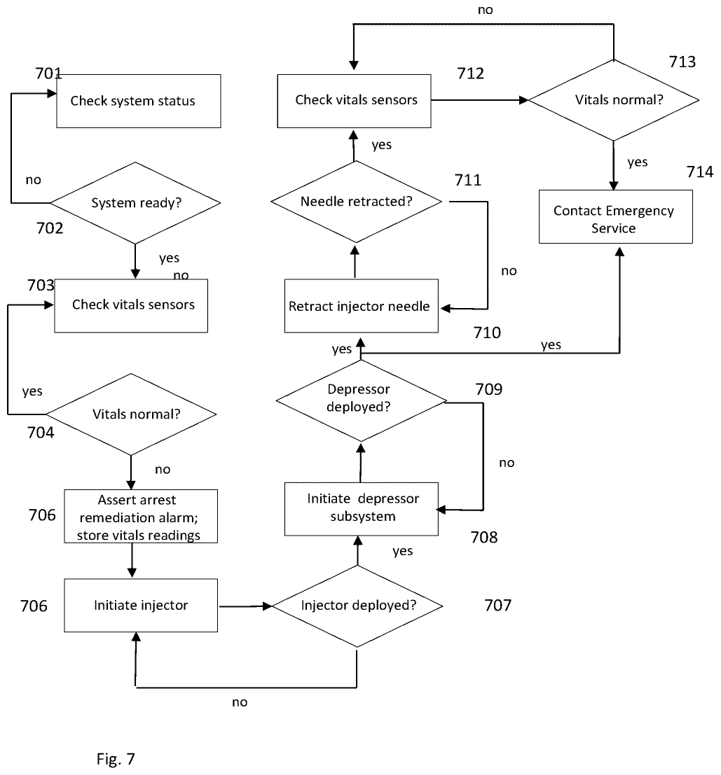

FIG. 7 shows a block diagram of a program portion that first determines if system is ready, 701 and 702, and, if ready, proceeds to check vital sensor inputs 703, and continues to do so as long as vital-sensor inputs fall within normal ranges 704. Should the sensor inputs deviate from normal ranges, the invention system's program asserts an arrest alarm 705 and stores those vital-sensor readings. Next, the program commands the injector subsystem to deploy 706 and checks for deployment 707 and the EMS system is called or otherwise communicated with. In some embodiments, the wearer may also initiate the injector. Once the injector is deployed, and the needle has penetrated the user's skin, the program commands the depressor subsystem to compress the drugs bladder or cartridge 708 and checks that compression has been deployed 709. The depressor may be embodied as an Archimedes device, according to some embodiments. The Archimedes device is loaded with the reversal drug and once turned, deploys the reversal drug. The Archimedes device is beneficial in reducing the length of the device. When checks show that injector has deployed and injection has deployed, the program instructs the injector subsystem to retract the injector needle 710 and checks for retraction 711. At this point, the program reverts to checking vitals 712 until 713 vitals return to normal range of values. Then, the program commands the system to cancel the arrest alarm 714.

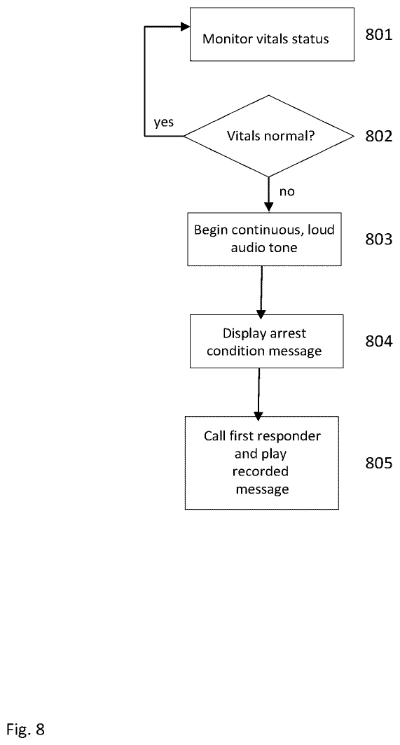

FIG. 8 shows a flow diagram for a program portion related to the arrest alarm. It commands the system to check vitals status 801 and continue to do so if normal 802. If values fall below a normal range of values 802, the program initiates 803 a continuous, loud piercing audio tone (arrest tone). Concurrently, the program commands that an appropriate message be displayed 804 and that a call is made to a first-responder phone number, using the cellular subsystem, and initiates playing a recorded message after the call is answered 805. The system may initiate the continuous audio tone (803) and call the first responder while communicating with a recorded message (805) simultaneously.

Tones, voice messages and displayed messages are stored as digital data in the microcontroller's data storage. When a system-status or arrest alert is invoked, the controller subsystem will convey stored data representing audio tones and voice messages to the transponder subsystem, and data representing graphical and textual messages to the display subsystem.

The invention system can be a lifeline for its user in the case of cardio-respiratory arrest. As such, it is important that the system be robust and failsafe. With electronic systems, based on semiconductor and electro-mechanical components, component conditioning and pre-production selecting reduces component failure to very low likelihood. Nevertheless, it is important that the system make use of its programmable functions to do frequent system self-checking to ensure that the system is highly likely to perform as designed.

With self-contained systems that rely on battery power, one point of potential failure is a battery that has discharged such that its voltage is approaching a level that renders operation unpredictable. The invention system herein disclosed makes use of programmed power-level checks to prevent the system from operating in an unpredictable way. Because of the purpose of the invention system, it is also critical that any reversal drug installed is not approaching its expiration date. Therefore, the use of coding on the side of the drug bladder or cartridge, and code reading by the invention system to capture that expiration data, helps ensure that as a reversal drug approaches expiration, the system will flag it, asserting a status alarm and placing the system in standby until the bladder or cartridge is replaced.

The figures and flow diagrams disclosed herein are exemplary. They should not be read as limiting the scope of the invention.

Size and materials are essentially non-critical. The encasement should be robust enough, and rigid enough, so that normal use will not allow crushing or vibratory shock to damage the system. In addition, the encasement should resist intrusion of moisture and heat. Electronic components and sensor, transducers, and the like may be discrete components or part of modular implementations.

The programmatic nature of the invention system would allow additional functions and features to be included without change to the underlying hardware infrastructure.

What is claimed is:

1. A method for detecting the onset of cardio-respiratory arrest and remediating the condition by automatic injection of reversal drugs comprising:
checking, by a controller subsystem, if a cardio-respiratory and remediating system is in ready status;
monitoring, by a controller subsystem, if system ready, vital-sign input data conveyed by one or more sensors;
comparing, by a controller subsystem, the vital-sign input data to predetermined normal value ranges;
continuing to monitor, by a controller subsystem, the vital-sign input data if within the predetermined normal value ranges;
asserting, by the controller subsystem, an arrest alarm if the vital-sign input data falls outside the predetermined normal value ranges;
initiating, by the controller subsystem, an injector subsystem to extend its hollow needle so as to penetrate a user's skin;
checking, by the controller subsystem, the injector subsystem status for needle extension;
initiating, by the controller subsystem, a depressor subsystem to compress a reversal-drug cartridge upon confirmation of the injector subsystem having extended its hollow needle;
checking, by the controller subsystem, the depressor subsystem status;
initiating, by the controller subsystem, a retraction to the injector system's hollow needle;
checking, by the controller subsystem, the injector subsystem status for retraction;
monitoring, by the controller subsystem, the vital-sign input data;
comparing, by the controller subsystem, the vital-sign input data to the predetermined normal value ranges;
continuing, by the controller subsystem, to monitor and compare the vital-sign input data;
cancelling, by the controller subsystem, the arrest alarm upon a return of the vital-sign input data values to within the predetermined normal value ranges.

2. The method as in claim 1 further comprising:
initiating, by a controller subsystem, after asserting the arrest alarm, a steady audible tone by the controller subsystem and transducer subsystem;
displaying, by a controller subsystem, a predetermined message on a display subsystem;
calling a first-responder phone number, by the controller subsystem and cellular communications subsystem; and
conveying, upon call pickup, a predetermined message including emergency situation and GPS coordinates, as conveyed to the controller subsystem by a GPS subsystem, to first-responder call recipient.

3. The method as in claim 1 further comprising:
checking, by the controller system, prior to checking if the cardio-respiratory and remediating system is in ready status, system power bus voltage level;
asserting a system status alarm if the system power bus voltage level is below a predetermined value; and
placing the system in standby;
continuing to check, by the controller system, the system power bus voltage level; and
cancelling the system status alarm and placing the system in ready mode if the system power bus voltage level is at or above the predetermined value.

4. The method as in claim 1 further comprising:
checking, by the controller system, prior to checking if the cardio-respiratory and remediating system is in ready status, an expiration date for the reversal-drug cartridge;
comparing, by a controller subsystem, the expiration date for the reversal-drug cartridge to a predetermined pre-expiration time period;
asserting, by a controller subsystem, a system status alarm if the expiration date falls with the pre-expiration time period;
placing, by a controller subsystem, the system in standby;
continuing to check, by the controller system, the expiration date; and
cancelling the system status alarm and placing the system in ready mode if a replacement reversal-drug cartridge now falls outside the pre-expiration time period.

5. The method as in claim 1 wherein:
asserting the arrest alarm comprises conveying a stored digital signal to the transducer subsystem resulting in a steady, piercing tone.

6. The method as in claim 1 wherein
asserting the arrest alarm comprises conveying a stored digital signal to the display subsystem resulting in display of any one or a combination of a graphic message, and a textual message.

7. The method as in claim 6 wherein:
the expiration date for the reversal-drug cartridge is presented in standard bar-code symbols.

8. The method as in claim 6 wherein:
comparing, by a controller subsystem, the expiration date for the reversal-drug cartridge to a predetermined pre-expiration time period is based on data conveyed to the controller subsystem by the code reader attached to the scaffold holding the reversal-drug cartridge.

9. The method as in claims 6 wherein:
asserting the arrest alarm comprises conveying a stored digital signal to the transducer subsystem resulting in beeping audio tone, and conveying a stored digital signal to the display subsystem resulting in display of one or a combination of a graphic message, and a textual message.
